(19) United States
(12) Patent Application Publication
(10) Pub. No.: US 2024/0050510 A1
(43) Pub. Date: Feb. 15, 2024

(54) COMBINATION THERAPY COMPRISING UNCARIA FOR TREATING ANXIETY AND DEPRESSION

(71) Applicant: The Open University, Raanana (IL)

(72) Inventor: Ravid DORON, Tel Aviv (IL)

(73) Assignee: THE OPEN UNIVERSITY, Raanana (IL)

(21) Appl. No.: 18/265,933

(22) PCT Filed: Dec. 9, 2021
(86) PCT No.: PCT/IL2021/051469

§ 371 Date: Jun. 7, 2023

(30) Foreign Application Priority Data
Dec. 10, 2020 (IL) 279372

(51) Int. Cl. A61K 36/74; A61K 31/343; A61K 31/138; A61K 31/55; A61K 31/496; A61P 25/24; A61P 25/22; A61K 36/734

(52) U.S. Cl. CPC A61K 36/74; A61K 31/343; A61K 31/138; A61K 31/55; A61K 31/496; A61P 25/24; A61P 25/22; A61K 36/734

(57) ABSTRACT

The present invention concerns methods and compositions employing a combination of Uncaria rhynchophylla (UR) herb and an antidepressant or anxiolytic drug therapy for treating or preventing anxiety, stress, depression, and/or symptoms thereof, wherein the combined administration of UR and the drug elicit a fast on-set response in the subject.

### TECHNOLOGICAL FIELD

The present disclosure relates to the use of the herb Uncaria rhynchophylla, as an accelerator of anxiolytic and antidepressant drugs in the treatment of anxiety and depression.

### BACKGROUND ART

References considered to be relevant as background to the presently disclosed subject matter are listed below:

[1] Yamaguchi, T., Tsujimatsu, A., Kumamoto, H., Izumi, T., Ohmura, Y., Yoshida, T., & Yoshioka, M. (2012). Anxiolytic effects of yokukansan, a traditional Japanese medicine, via serotonin 5-HT 1A receptors on anxiety related behaviors in rats experienced aversive stress. Journal of ethnopharmacology, 143(2), 533-539.
[2] Mizoguchi, K., Tanaka, Y., & Tabira, T. (2010). Anxiolytic effect of a herbal medicine, yokukansan, in aged rats: involvement of serotonergic and dopaminergic transmissions in the prefrontal cortex. Journal of ethnopharmacology, 127(1), 70-76.
[3] Shoji, H., & Mizoguchi, K. (2013). Brain region-specific reduction in c-Fos expression associated with an anxiolytic effect of yokukansan in rats. Journal of ethnopharmacology, 149(1), 93-102.
[4] Schwartz, K., Weizman, A. and Rehavi, M. (2005) The effect of psychostimulants on [3H] dopamine uptake and release in rat brain synaptic vesicles. J. Neural Trans. 113: 1347-1352.
[5] Gurwitz, D. and Rehavi, M. (2005) Pharmacogenetics: towards personalized medicine. Harefua. 144: 711-716.
[6] Barkan, T., Peled, A., Modai, I., Weizman, A. and Rehavi, M. (2006) Characterization of the serotonin transporter in lymphocytes and platelets of schizophrenia patients treated with atypical or typical antipsychotics compared to healthy individuals. Eur. Neuropsychopharmacol. 16: 429-436.
[7] Schwartz, K., Herman, I., Peer, G., Weizman, A., and Rehavi, M. (2006) Elevated platelet vesicular monoamine transporter 2 in former heroin addicts maintained on methadone. J. Neural . Transm. 114: 281-284.
[8] Barkan T., Pelled A., Modai, I., Weizman, A. and Rehavi, M. (2006) Serotonin transporter characteristics in lymphocytes and platelets of male aggressive schizophrenia patients compared to non-aggressive schizophrenia patients. Eur. Neuropsychopharmacol. 16: 572-579.
[9] Schwartz, K., Nachman, R., Yossifoff m., Sapir, R., Weizman, A. and Rehavi, M. (2007) Cocaine, but not amphetamine, short term treatment elevates the density of the vesicular monoamine transporter 2 in rat brain. J. Neural. Transm. 114: 427-430.
[10] Doron, R., Lotan, D., Einat, N., Yaffe, R., Winer, A., Marom, I., . . . & Rehavi, M. (2014). A novel herbal treatment reduces depressive-like behaviors and increases BDNF levels in the brain of stressed mice. Life sciences, 94(2), 151-157.
Acknowledgement of the above references herein is not to be inferred as meaning that these are in any way relevant to the patentability of the presently disclosed subject matter.

### BACKGROUND

Depression and anxiety disorders are highly prevalent and considered of major public health concern worldwide. Despite the availability of a wide range of drugs for treating depression and anxiety, most patients fail to achieve complete and sustained remission of symptoms. Selective serotonin reuptake inhibitors (SSRIs) and serotonin-norepinephrine reuptake inhibitors are the current first-line treatment for depression and anxiety, although they have some pronounced limitations. Most notably, they are characterized by a low success rate and slow-onset and are associated with a wide variety of side effects, including sexual dysfunction, weight gain, sleep disruption and gastrointestinal symptoms. Adverse side effects and delayed therapeutic effect may lead to discontinuation of treatment. Moreover, due to the low efficacy and slow- onset nature of these drugs, many patients may experience long periods of depressive and anxious symptoms without being beneficially treated. This period might be highly critical for suicide-risk patients.

Uncaria rhynchophylla (UR) herb is one of the herbs included in the traditional Japanese medicine Yokukansan which has been shown to have anxiolytic properties [1-3]. Acute or sub-chronic administration of UR induced an anxiolytic-like effect which was reversed by blockade of 5-HT1A receptors [4]. Also, treatment with UR or its active constituent geissoschizine methyl ether (GM) ameliorated isolation-induced increased aggressiveness and decreased sociality [5], which are linked to disturbances in the serotonergic system [6-7]. Another active constituent of UR which is associated with antidepressant-like activity is rhynchophylline (RHY) [8].Combination of RHY with conventional antidepressants such as fluoxetine and imipramine led to augmentation of the antidepressant-like effect [9].

There is a great demand for novel antidepressant and anxiolytic treatments with fast-onset, sustained efficacy, and minimal side effects. Thus, there is a need for an accelerator of anxiolytic and antidepressant drugs.

### GENERAL DESCRIPTION

In a first of its aspects, the present invention provides an Uncaria rhynchophylla (UR) herb for use in a method of treating or preventing anxiety, stress, depression, and/or symptoms thereof in a subject in need thereof, said method comprising conjointly administering the Uncaria rhynchophylla (UR) herb and at least one antidepressant or anxiolytic drug, or an antidepressant or anxiolytic therapy to said subject.

In another aspect, the present invention provides a method of treating or preventing anxiety, stress, depression, and/or symptoms thereof, said method comprising conjointly administering an Uncaria rhynchophylla (UR) herb and at least one antidepressant or anxiolytic drug, or an antidepressant or anxiolytic therapy to a subject in need thereof.

In another aspect, the present invention provides a method of enhancing the rate at which an antidepressant or anxiolytic drug, or an antidepressant or anxiolytic therapy affects the symptoms of anxiety, stress, and/or depression in a subject, said method comprising conjointly administering an Uncaria rhynchophylla (UR) herb together with said antidepressant or anxiolytic drug, or said antidepressant or anxiolytic therapy to a subject in need thereof.

In one embodiment, said stress is chronic stress or acute stress.

In one embodiment, said depression is Major Depressive Disorder (MDD).

In one embodiment, said administration is during a mild, moderate, or severe depressive episode.

In one embodiment, the UR is administered orally, topically, by rectal administration or by injection.

In one embodiment, the UR is provided as capsulated powder, tablet, pellets, dissolved in a suitable fluid, ointment, cream, or suppositories.

In one embodiment, the UR is capsulated in a 0.5 gram capsule.

In one embodiment, the UR is administered in an amount of 450 mg per day, 0.5 g per day, 1 g per day, 2 g per day or more.

In one embodiment, the UR is administered once a day, twice a day, three times a day or more.

In one embodiment, said antidepressant or anxiolytic drug is selected from the group consisting of a serotonin modulator, a Selective serotonin reuptake inhibitor (SSRI), a serotonin-norepinephrine reuptake inhibitor, an herbal composition, and a combination thereof.

In one embodiment, said serotonin modulator is vilazodone.

In one embodiment, said Selective serotonin reuptake inhibitor is any one of escitalopram, fluoxetine, or sertraline.

In one embodiment, said serotonin-norepinephrine reuptake inhibitor is imipramine. In one embodiment, said herbal composition is selected from a group consisting of Shan Zha, an active fraction of Shan Zha, and a combination of hawthorn fruit (Shan Zha), light wheat grain (Fu xiao mai), Lilly Bulb (bai hi) and Chinese date (Da zao).

In one embodiment, said conjoint administration comprises administering the UR and the at least one antidepressant or anxiolytic drug in a single composition or in separate compositions.

In one embodiment, said antidepressant or anxiolytic therapy comprises psychotherapy, cognitive behavioral therapy (CBT), ECT (electroconvulsive therapy), transcranial direct current stimulation (tDCS), transcranial magnetic stimulation (TMS), DBS (deep brain stimulation), or physical exercise.

In one embodiment, said conjoint administration reduces the time required for reduction of anxiety, stress and/or depression symptoms.

In one embodiment, said symptoms are evaluated using a Symptoms Check List (SCL)-90, Clinical Global Impression (CGI), Hamilton anxiety rating scale (HAM-A), Hamilton depression rating scale (HAM-D), Sheehan Disability Scale (SDS), or visual analogue scales (VAS).

In one embodiment, said VAS are general anxiety or mood.

In one embodiment, said conjoint treatment is for 1 day, 2 days, 3 days, 4 days, 5 days, 6 days, 1 week, 2 weeks, 3 weeks, 4 weeks, 5 weeks, 6 weeks or more.

In one embodiment, said conjoint treatment does not cause weight gain, reduced sexual function, and/or reduced memory function.

In another aspect, the present invention provides an Uncaria rhynchophylla (UR) herb for use in a method of treating or preventing anxiety, stress, depression, and/or symptoms thereof with reduced side effects in a subject in need thereof, said method comprising conjointly administering the Uncaria rhynchophylla (UR) herb and at least one antidepressant or anxiolytic drug or an antidepressant or anxiolytic therapy to said subject.

In one embodiment, said side effects are one or more of weight gain, reduced sexual function, and/or reduced memory function.

In another aspect, the present invention provides a composition comprising Uncaria rhynchophylla (UR) herb and at least one physiologically acceptable carrier, diluent, excipient and/or additive for use in a method of treating or preventing anxiety, stress, depression, and/or symptoms thereof in a subject in need thereof, said method comprising conjointly administering said composition and at least one antidepressant or anxiolytic drug or an antidepressant or anxiolytic therapy to said subject.

In another aspect, the present invention provides a composition comprising Uncaria rhynchophylla (UR) herb and at least one antidepressant or anxiolytic drug, said composition optionally further comprising at least one physiologically acceptable carrier, diluent, excipient and/or additive.

In one embodiment, said composition is for use in treating or preventing anxiety, stress, depression, and/or symptoms thereof.

In certain embodiments the composition of the invention is a pharmaceutical composition or herbal supplement.

In another aspect, the present invention provides a kit comprising Uncaria rhynchophylla (UR) herb and at least one antidepressant or anxiolytic drug, said kit optionally further comprising instructions for conjoint use of the UR and the at least one antidepressant or anxiolytic drug in treating or preventing anxiety, stress, depression, and/or symptoms thereof.

In another aspect, the present invention provides an Uncaria rhynchophylla (UR) herb for use in a method of rapid-onset treatment or prevention of anxiety, stress, depression, and/or symptoms thereof, said method comprising administering the Uncaria rhynchophylla (UR) herb as a single agent.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1D: The effect of 1-week combination treatments of NHT or escitalopram with UR on anxiety-like behavior.

FIG. 1A: Time in open arms (sec) following treatment with UR in combination with NHT (Novel Herbal Treatment—a herbal composition comprising four components: hawthorn fruit (Shan Zha), light wheat grain (Fu xiao mai), Lilly Bulb (bai hi) and Chinese date (Da zao)) and escitalopram compared with control (saline).

FIG. 1B: Time in open arms (sec) following treatment with UR in combination with NHT, Shan-zha, fluoxetine or sertraline compared with control (saline).

FIG. 1C: One-week combined treatments of UR with NHT and escitalopram induced a fast-onset anxiolytic effect in the OFT (CIT=escitalopram, Vehicle=saline).

FIG. 1D: Three-week combined treatments induced sustained anxiolytic effect in the OFT (CIT=escitalopram, Vehicle=saline).

Results are presented as mean±SEM *p<0.05, **p<0.005 vs. the vehicle, saline (n=13-15).

FIG. 2: The effect of 3-week combination treatments of NHT or escitalopram with UR on anxiety-like behavior.

Either combined UR treatments with NHT and escitalopram or individual treatments increased the time spent in the open arms of the EPM compared to saline (n=13-14).

FIG. 3A-3B: The effect of 1- and 3-week combination treatments of NHT or escitalopram with UR on hippocampal BDNF levels.

FIG. 3A: At 1 week 1, combined UR treatments with NHT and escitalopram increased hippocampal BDNF levels while individual treatments reduced its levels compared to saline, (n=10-11).

FIG. 3B: At 3 weeks, NHT and escitalopram individual treatments increased BDNF levels compared to saline, (n=10-11).

FIG. 4A-4B: Effect of treatment in the novel open field test. Effect of treatment with NHT (30 mg/kg) n=15, Escitalopram (CIT) (15 mg/kg) n=15, UR (15 mg/kg) n=15, NHT (30 mg/kg)+UR (15 mg/kg) n=15, Escitalopram (15 mg/kg)+UR (15 mg/kg), Shan Zha (30 mg/kg)+UR (15 mg/kg) n=15, Saline (30 mg/kg) n=15] on anxiety-like behavior in the novel open field test following one week of treatment (A) and three weeks of treatment (B). * p<0.05. **p<0.01.

FIG. 5A-5B: Effect of treatment in the Forced Swim Test.

Effect of treatment with NHT (30 mg/kg) n=15, Escitalopram (CIT) (15 mg/kg) n=15, UR (15 mg/kg) n=15, NHT (30 mg/kg)+UR (15 mg/kg) n=15, Escitalopram (15 mg/kg)+UR (15 mg/kg), Shan Zha (30 mg/kg) +UR (15 mg/kg) n=15, Saline (30 mg/kg) n=15 on depressive-like behavior in the Forced Swim Test following one week of treatment (A) and three weeks of treatment (B). *p<0.05. **p<0.01.

FIG. 6A-6B: Sexual Behavior in mice treated with escitalopram or Shan-zha in combination with UR.

FIG. 6A: Stressed male mice were given either (CIT) (n=15) or escitalopram+UR (n=15) for 1 week, and then their sexual behavior was tested.

FIG. 6B: Stressed male mice were given either Shan-zha (n=15) or Shan-zha+UR (n=15) for 1 week, and then their sexual behavior was tested.

Data shown in Mean+/−SEM and compared using Two-tailed T-test [p=0.0016 and p=0.13, respectably).

FIG. 7A-7B: Measurement of Uncaria-evoked GIRK currents at −80 mV.

FIG. 7A: A Graph showing the K+ current in a GIRK expressing oocyte presented as juA per second. The initial culture solution (ND96) was replaced with a high K+ solution (24 mMK+) thereby inducing a K+ current, further application of Uncaria induced an additional current. The current disappeared upon replacement to the ND96 solution.

FIG. 7B. A Graph showing current (I, in nA)-voltage (V, in mV) relationship measured from the same oocyte before (representing the basal K+ current through the GIRK channel, shown in black) and after the application of Uncaria (representing the total current through the GIRK channel after the application of Uncaria).

### DETAILED DESCRIPTION OF EMBODIMENTS

The present disclosure is based on the surprising finding that co-administration of the herb Uncaria rhynchophylla (UR) with antidepressant or anxiolytic drugs, which typically have a relatively slow-onset effect, was successful in achieving rapid and persistent amelioration of depression and anxiety symptoms at an earlier time point than otherwise expected. Namely, as shown in the examples below, an improvement in various physiological and behavioral parameters was evident as early as one week after the combined treatment has commenced, in animal models of anxiety and depression. At this early time point, treatments with antidepressant or anxiolytic drugs alone (i.e., as a single agent) were ineffective. The present disclosure therefore proposes a combination therapy comprising Uncaria and at least one anti-depressive and/or antianxiolytic drug, or an antidepressant or anxiolytic therapy.

In a first of its aspects, the present invention provides an Uncaria rhynchophylla (UR) herb, or a composition comprising UR, for use in a method of treating or preventing anxiety, stress, depression, and/or symptoms thereof in a subject in need thereof, said method comprising conjointly administering the Uncaria rhynchophylla (UR) herb and at least one antidepressant or anxiolytic drug, or an antidepressant or anxiolytic therapy to said subject.

In another aspect, the present invention provides a method of treating or preventing anxiety, stress, depression, and/or symptoms thereof, said method comprising conjointly administering an Uncaria rhynchophylla (UR) herb or a composition comprising UR, and at least one antidepressant or anxiolytic drug, or an antidepressant or anxiolytic therapy to a subject in need thereof.

In another aspect, the present invention provides a method of enhancing the rate at which an antidepressant or anxiolytic drug, or an antidepressant or anxiolytic therapy affects the symptoms of anxiety, stress, and/or depression in a subject, said method comprising conjointly administering an Uncaria rhynchophylla (UR) herb or a composition comprising UR, together with said antidepressant or anxiolytic drug, or said antidepressant or anxiolytic therapy to a subject in need thereof.

In one particularly preferred embodiment of the invention, the combination therapy of the invention reduces the timeframe (in other words, minimizes the time lag) in which the anti-depressive and/or antianxiolytic drug or therapy exert their beneficial effect.

As used herein, the term “ [symbol] ” (also referred to herein as “ [symbol] ”) refers to natural plant material (also termed cat's claw herb). Any effective part of the herb in accordance with the present invention can be used (for example, crude, purified or partially purified extracts in a form of e.g., a powder), including seeds, leaves, stems, flowers, roots, fruit, bark, or any other plant parts which are useful for the purposes described, particularly the herb's hooks (also referred to as spikes or tendrils).

As used herein the term “ [symbol] ” refers to a composition comprising or consisting essentially of UR as the active ingredient in an amount effective for enhancing the rate at which an antidepressant or anxiolytic drug, or an antidepressant or anxiolytic therapy affects the symptoms of anxiety, stress, and/or depression in a subject in need thereof.

Such compositions may be provided as pharmaceutical compositions, nutraceutical compositions, compositions for use in addition to food and beverage products, and so on.

The term “ [symbol] ” or “ [symbol] ” is used conventionally and refers to the management or care of a subject for the purpose of preventing, combating, alleviating, reducing, relieving, or improving a subject's anxiety, stress, depression, or any symptom thereof. The term encompasses any reduction in the subject's anxiety, stress, and/or depression as evidenced, for example, by a subject's personal report, by suitable questionnaires, or by measurement of physiological indicators of anxiety e.g., blood cortisol levels, whereby high levels of cortisol are indicative of stress.

As used herein, “ [symbol] ” or providing a “ [symbol] ” (to prevent) or a “ [symbol] ” is acting in a protective manner, to defend against or prevent something, especially a condition or disease.

As used herein the term “ [symbol] ” refers to different forms of abnormal and pathological fear and anxiety. The term encompasses anxiety disorders characterized by continuous or episodic symptoms including generalized anxiety, phobic, and panic disorders. Anxiety disorders are characterized by mental apprehension, and various physical symptoms such as physical tension.

The term “ [symbol] ”, as used herein, refers to a mental state of depressed mood characterized by feelings of sadness, despair, and discouragement. In some instances, depression is a clinical symptom, and can include, but not limited to, Major Depressive Disorder (including single episode and recurrent), unipolar depression, treatment-refractory depression, resistant depression, anxious depression, and dysthymia (also referred to as dysthymic disorder). Further, the term “depression” can encompass any major depressive disorder, dysthymic disorder, mood disorders due to medical conditions with depressive features, mood disorders due to medical conditions with major depressive-like episodes, substance-induced mood disorders with depressive features and depressive disorder not otherwise specific as defined by their diagnostic criteria.

As used herein the term “ [symbol] ” encompasses both chronic and acute stress conditions. As used herein, “ [symbol] ” (MDD) or “Major depression disorder” or “unipolar disorder” refers to a mood disorder involving any of the following symptoms: persistent sad, anxious, or “empty” mood; feelings of hopelessness or pessimism; feelings of guilt, worthlessness, or helplessness; loss of interest or pleasure in hobbies and activities that were once enjoyed, including sex; decreased energy, fatigue, being “slowed down”; difficulty concentrating, remembering, or making decisions; insomnia, early-morning awakening, or oversleeping; appetite and/or weight loss or overeating and weight gain; thoughts of death or suicide or suicide attempts; restlessness or irritability; or persistent physical symptoms that do not respond to treatment, such as headaches, digestive disorders, and chronic pain. The term “Major Depressive Disorder” (MDD) is understood in art and refers to a diagnosis that is guided by diagnostic criteria listed in Diagnostic and Statistical Manual of Mental Disorders, 5th Edition (DSM-5) or any later edition thereof, or the World Health Organization's International Statistical Classification of Diseases and Related Health Problems (ICD-10).

The present invention also encompasses the treatment or prevention of menopause symptoms such as, but not limited to, mood changes and hot flashes.

The UR herb or the compositions of the invention can be administered and dosed by the methods of the invention, in accordance with good medical practice. For example, the UR herb or the composition can be administered by any suitable route including but not limited to intraperitoneal, subcutaneous, transcutaneous (transdermal), topical, intramuscular, intraarticular, subconjunctival, rectal, vaginal, mucosal, e.g., oral (including buccal or sublingual), intranasal, or intraocular administration.

In yet some further embodiments, the composition of the invention may optionally further comprise at least one of physiologically acceptable carrier/s, excipient/s, additive/s diluent/s and adjuvant/s.

More specifically, compositions used to treat subjects in need thereof according to the invention, which may conveniently be presented in unit dosage form, may be prepared according to conventional techniques well known in the pharmaceutical and nutraceutical industry. Such techniques include the step of bringing into association the active ingredients with the pharmaceutically or physiologically acceptable carrier(s) or excipient(s). In general, compositions are prepared by uniformly and intimately bringing into association the active ingredients of the invention with liquid carriers or finely divided solid carriers or both, and then, if necessary, shaping the product.

The compositions may be formulated into any of many possible dosage forms such as, but not limited to, tablets, capsules, liquid syrups, soft gels, suppositories, and enemas. In one embodiment, the UR is capsulated in a 0.1-1 gram capsule, or 0.2 gram capsule, 0.3 gram capsule, or 0.4 gram capsule, or 0.5 gram capsule, or 0.6 gram capsule, or 0.7 gram capsule, or 0.8 gram capsule, or 0.9 gram capsule, or 1 gram capsule. It is appreciated that capsules of higher UR content may also be formulated.

The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In addition to the ingredients particularly mentioned above, the compositions may also include other agents conventional in the art having regard to the type of composition in question. For example, the compositions of the invention may be incorporated into food products, beverages (e.g., juices) or combined with commonly used food additives such as corn syrup.

Still further, pharmaceutical compositions are compositions that include pharmaceutically acceptable vehicle. “ [symbol] [symbol] ” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable, or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating, and coloring agents may be used. Pharmaceutical compositions may be formulated into preparations in solid, semisolid, liquid, or gaseous forms, such as tablets, capsules, powders, granules, ointments, solutions, suppositories, injections, inhalants, gels, microspheres, and aerosols.

Still further, the composition/s of the invention may be applied as a single daily dose or multiple daily doses. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month.

The application of the conjoint treatment of the invention may last up to a day, two days, three days, four days, five days, six days, 1 week, 2 weeks, 3 weeks, 4 weeks, 5 weeks, 6 weeks, a month, two months three months or even more. In one embodiment, application may last from one week to one month.

As used herein the terms “ [symbol] ” or “ [symbol] ” refer to a combined administration or treatment. Specifically, in the context of the present invention these terms refer to the combined administration of Uncaria rhynchophylla (UR) herb with one or more antidepressant or anxiolytic drugs or therapies. “ [symbol] ” encompasses both simultaneous administration of UR and the drug or therapy (either in the same composition or in separate compositions), and sequential administration whereby UR may be administered either immediately prior to or immediately after the administration of the additional drug or therapy.

As indicated above, the present invention concerns the conjoint administration of Uncaria rhynchophylla (UR) herb with one or more antidepressant or anxiolytic drug. As used herein the term “ [symbol] [symbol] ” refers to any drug that is known to affect the symptoms of anxiety, stress, and/or depression. Non-limiting examples include serotonin modulators e.g., vilazodone, selective serotonin reuptake inhibitors (SSRIs) e.g., escitalopram (Cipralex), fluoxetine (Prozac), sertraline, citalopram, dapoxetine, fluvoxamine, paroxetine (Seroxat), and vortioxetine (Brintellix); serotonin-norepinephrine reuptake inhibitors (SNRIs) e.g., atomoxetine, desvenlafaxine, duloxetine, levomilnacipran, milnacipran, sibutratnine, tramadol, venlafaxine; noradrenaline and specific serotonergic antidepressants (NASSAs) e.g., mirtazapine; tricyclic antidepressants (TCAs) e.g., amitriptyline, clomipramine, dosulepin, imipramine, lofepramine, nortriptyline; serotonin antagonists and reuptake inhibitors (SARIs) trazodone; monoamine oxidase inhibitors (MAOIs) e.g., tranylcypromine, phenelzine, isocarboxazid.

In one embodiment the antidepressant or anxiolytic drug is a serotonin modulator. In a specific embodiment the serotonin modulator is vilazodone.

In a specific embodiment the antidepressant or anxiolytic drug is a selective serotonin reuptake inhibitor (SSRI). In another specific embodiment the SSRI is one or more of escitalopram (Cipralex), fluoxetine (Prozac), or sertraline.

In another specific embodiment the antidepressant or anxiolytic drug is imipramine.

The term “ [symbol] [symbol] ” also encompasses herbal drugs e.g., shan-zha or a fragment thereof or herbal compositions comprising combinations of several herbs, e.g., herbal compositions comprising shan-zha, light wheat grain, Lilly bulb and Chinese date (also termed herein New Herbal Treatment (NHT)), as described in WO 2011/111039.

As used herein the term “ [symbol] ” (also termed herein hawthorn plant or Crataegus pinnatifida) refers to dry extract of the plant's fruit. Hawthorn (also termed Crataegus pinnatifida or Shan Zha) fruit contains high levels of triterpene acids and the flavonoids hyperoside and isoquercitrin (Cui et al., 2006a), the active constituents that determine its physiological effects. Both flavonoids and triterpenes belong to the two main groups of adaptogen active elements.

Accordingly, as used herein the term a “ [symbol] ” or a “ [symbol] [symbol] ”, refers to fractions of Shan-zha which were obtained by subjecting Shan-zha to various ethanol concentrations, for example, but not limited to 10%, 20%, 50% or 70% ethanol. Particularly, 20%, 50% or 70% ethanol. Exemplary Methods for preparation of a Shan-Zha fraction are disclosed in PCT/IL2021/050685.

As used herein, “ [symbol] ” also referred to Fu xiao mai or Triticum aestivum is a cultivated wheat species. Aqueous acid extracts of wheat grain were found to contain a series of compounds belonging to the classical 5-phenyl-1, 4-benzodiazepinones which display high affinity to the central benzodiazepine receptor in the brain (Wildmann et al., 1988). One of the compounds was shown to be identical to diazepam, which mediates anxiolytic and muscle relaxant effects by acting on inhibitory GABAergic receptors (Chouinard, 2004). Animal studies support a hypnotic effect in rats that were treated with an herbal formula containing Triticum aestivum, Radix glycyrrhizae and Fructus ziziphus.

As used herein, “ [symbol] ” (also termed Lilium brownii or Bai he) is an adaptogen which exhibits high inhibitory activity and selectivity towards monoamine oxidase (MAO)-B, which catalyzes the oxidative deamination of biogenic monoamines, in rat brain homogenates (Lin et al., 2003). Inhibition of MAO-B elevates monoamine levels, which is the basis for the action of anti-depressant and anxiolytic treatments in the clinic, such as MAO inhibitors and SSRIs (Sheehan and Sheehan, 2007). In addition, selective MAO-B inhibitors, unlike non-selective MAOIs, do not cause tyramine accumulation and thus do not induce the dangerous hypertensive crisis (also known as the “cheese effect”) (Sheehan and Sheehan, 2007). Studies show that lilium exerts immunomodulatory functions and may suppress overreactive immune responses (Ko and Leung, 2007). It also possesses antioxidant properties.

As used herein, “ [symbol] ” or “ [symbol] ” (also termed Fructus Zizyphi Jujubae) has been used in traditional medicine to treat insomnia and anxiety. In modern pharmacological studies, Ziziphi Jujuba was shown to possess hypnotic-sedative, hypertensive, anti-hypoxia, and hypothermic effects.

In specific embodiments the combination of treatments in accordance with the present invention comprises a combination of escitalopram and UR, fluoxetine and UR, sertraline, and UR, vilazodone and UR, NHT and UR, or Shan-Zha (SZ) and UR.

As used herein the term “ [symbol] [symbol] ” refers to any non-pharmacological therapy that is known to affect the symptoms of anxiety, stress, and/or depression. Non-limiting examples include psychotherapy, cognitive behavioral therapy (CBT), brain stimulation e.g., ECT (electroconvulsive therapy), transcranial direct current stimulation (tDCS), transcranial magnetic stimulation (TMS), or DBS (deep brain stimulation), and life-style modifications such as physical exercise.

In some embodiments the composition of the invention may comprise additional active ingredients appropriate for the treatment of anxiety, stress, or other conditions. Such active ingredients may be obtained from herbs, minerals and other ingredients having calming or anxiolytic effects including, but not limited to: Zhu Sha (cinnabaris), Ci Shi (magnetitum), Long Gu (os draconis) , Long Chi (dens draconis) , Mu Li (concha ostreae) , Hu Po (succinum) , Zhen Zhu (margarita) , Zhen Zhu Mu (concha margaritaferae) , Suan Zao Ren (semen zizyphi spinosae), Bai Zi Ren (semen platycladi), Yuan Zhi (radix polygalae), He Huan Pi (cortex albiziae), Ye Jiao Teng (caulis polygoni multiflori), Ling Zhi (ganoderma), Dan Shen (radix salviae miltiorrhizae), Fu Shen (poria paradicis), Lian Zi (semen nelumbinis), Long Yan Rou (arillus longan), Mai Men Dong (radix ophiopogonis), Ren Shen (radix ginseng) , Shi Chang Pu (rhizoma acori), Wu Wei Zi (fructus schisandrae chinensis), Hong zao (Red date).

In certain embodiments, the composition of the invention may further comprise additional active agents such as antioxidants (e.g., selenium), vitamins (such as vitamin A, B1, B2, thiamine, B6, pyridoxine, B complex, biotin, nicotinic acid, B12, C, ascorbic acid, D, D2, D3, E, riboflavin, K, K1 or K2), Co Enzyme Q10, NADH, NAD, D-ribose, or amino acids such as L-Glutamine or Lysine.

In another aspect, the present invention provides an Uncaria rhynchophylla (UR) herb for use in a method of rapid-onset treatment or prevention of anxiety, stress, depression, and/or symptoms thereof, said method comprising administering the Uncaria rhynchophylla (UR) herb as a single agent. As used herein the term “ [symbol] ” is wherein the onset of the antidepressant or anxiolytic activity of UR occurs very early upon administration, e.g., after 1 day, or 2 days, or 3 days, or 4 days, or 5 days, or 6 days or one week after initial administration.

Interestingly, as shown in Example 7 below, Uncaria appears to induce activation of certain GIRK channels.

The following examples are representative of techniques employed by the inventors in carrying out aspects of the present invention. It should be appreciated that while these techniques are exemplary embodiments for the practice of the invention, those of skill in the art, in view of the present disclosure, will recognize that numerous modifications can be made without departing from the spirit and intended scope of the invention.

### EXAMPLES

### Experimental Procedures

Animals. ICR outbred mice (Envigo RMS (Harlen), Israel) were kept in the vivarium of the Psychobiological laboratory of the Open University, Hadassah Ein Karem Medical Center, Jerusalem. Mice were kept on a reversed 12-hour light/dark cycle and given ad libitum access to food and water. All experiments were performed during the dark phase under red light (7:00-19:00). All experiments were approved by the Committee for animal care and use according to the NIH guidelines.

Drugs administration. Uncaria rhynchophylla (UR) herb, Crataegus pinnatifida (hawthorn fruit (Shan Zha)), Triticum aestivu (light wheat grain (Fu xiao mai)), Lilium brownie (Lilly Bulb (bai he)), and the fruit of Ziziphus jujuba (Chinese date (Da zao) were purchased as freeze-dried granules from KPC Products, Inc (Irvine, CA, USA). Escitalopram was kindly donated by TEVA Ltd (Israel). The drugs were dissolved in saline and daily administered intraperitoneally in the following doses: UR 15 mg/kg, escitalopram 15 mg/kg, fluoxetine, and sertraline 20 mg/kg, Shan-zha 30 mg/kg. The drugs were administered simultaneously in two separate injections; the first containing a drug selected from SSRIs, Shan-zha, NHT (New Herbal Treatment—a herbal composition comprising four components: hawthorn fruit (Shan Zha), light wheat grain (Fu xiao mai), Lilly Bulb (bai hi) and Chinese date (Da zao)), vilazodone or saline and the second containing UR or saline.

Unpredictable chronic mild stress (UCMS). Mice were exposed to UCMS for 4 weeks at the age of 4 weeks as previously described (Malkoff, A., et al. (2008) J Neural Transm 115, 1563-1571).

Elevated plus-maze (EPM) and Open field test (OFT). Anxiety-like behavior was assessed using EPM and OFT as previously described (Eliash, S., Det al. (2009) Brain Res 1256, 138-148). Locomotion was assessed using the OFT and expressed as the percentage of time that the mouse moved in a velocity above 0.1 pixel/sec.

Tall Suspension test (TST). Depressive-like behavior was assessed using TST as previously described (Malkoff, A., et al. (2008) J Neural Transm 115, 1563-1571).

Y maze and Object recognition test (ORT). Short-term and long-term memory were evaluated using the Y maze and ORT, respectively, as previously reported (Schreiber, S., et al. (2014) Behavioral brain research, 271,59-64).

Body weight monitoring. Body weight was monitored every 3 days throughout UCMS and treatment.

Evaluation of sexual function. Number of mounts was recorded as previously described (Nomura, M., et al. (2007) Physiology & behavior, 91(2), 223-228). A female mouse in estrus was placed in the male's home cage during the dark phase under red dim light for the duration of 30 min. Male sexual behavior, including number of mounts with or without intromission and latency to first mount was recorded.

Novel Open Field (NOF). The novel open field consists of an empty square arena (40×40×40 cm) divided into 36 identical squares and surrounded by Perspex opaque walls. This test reflects the conflict between the innate fear that mice have of the central area of a novel open field versus their desire to explore new environments. When anxious, the natural tendency of mice is to prefer staying close to the walls. Mice were placed in the center of the arena and their behavior was video recorded for a total of 5 min, and later coded by an observer blind to the mice's treatment. Anxiety-like behavior was measured by the time spent in the peripheral area, i.e., the peripheral ring of the arena (6.67 cm from each wall, Walsh & Cummins, (1976) Psychological Bulletin, 83 (3), 482-504). The arena was thoroughly cleaned with ethanol and allowed to dry between subjects in order to eliminate any odor cues. Effects were evaluated in independent mice groups following 1 and 3 weeks of treatment. Test was record by Viewer III (BIOBSERVE) software.

Forced Swim Test (FST). Animals were tested in the FST following the method described by Porsolt et al. (Nature (1977) 266, 730-732). The FST possesses high predictive validity of depressive behavior (Willner, Psychopharmacology (1984) 83 (1) 1-16). Swimming sessions were conducted by placing mice in individual glass cylinders (60 cm high×25 cm in diameter) containing 25 cm deep water at a temperature of 23° C., such that the mice were not able to support themselves by touching the bottom with their feet. The swimming sessions were performed between 12:00 to 19:00 o'clock. Two swimming tests were performed in parallel in each examination session. The water was thoroughly changed after each 10 mice and was cleaned between subjects to eliminate any odor cues. Depression like behavior was measured as the time the animal spent floated without moving, and swimming was defined as the time the animal spent engaged in active swimming or struggle movement. Effects were evaluated in independent mice groups following 1 and 3 weeks of treatment. The test was recorded by Viewer III (BIOB SERVE) software.

Data analysis and interpretation of results. Data was analyzed using a one-way ANOVA with Treatment as a between subject variable, performed on the time spent in the center area in the Novel Open Field and the time spent in the open arms for anxiety like behavior, and depressive like behavior was measured as the time the animal spent floating without moving. Where results were significant, the ANOVA was followed by a planned contrasts analysis by one-way DUNNETT test. This test was chosen to compare the significance of each treatment group as compared with the Saline group. Significance was assumed as p<0.05.

Assessment of BDNF levels. Hippocampus tissue samples are prepared as previously described (Maayan, R., et al., European neuropsychopharmacology, 2006. 16(5): p. 329-339). Briefly, tissues were homogenized in cold extraction Tris-buffer and then acidified with 0.1 M HCl (pH 3.0) for 15 min at room temperature. Homogenates were neutralized with 0.1 M NaOH (pH 7.6) and centrifuged in a microcentrifuge at 7,000 g for 10 min. BDNF levels in the supernatant were evaluated using sandwich ELISA as previously described (Baker-Herman, T. L., et al., Nat Neurosci, 2004. 7(1): p. 48-55).

### Example 1

### Alterations in BDNF Levels in Mice Treated With a Combination of UR with Escitalopram or NHT

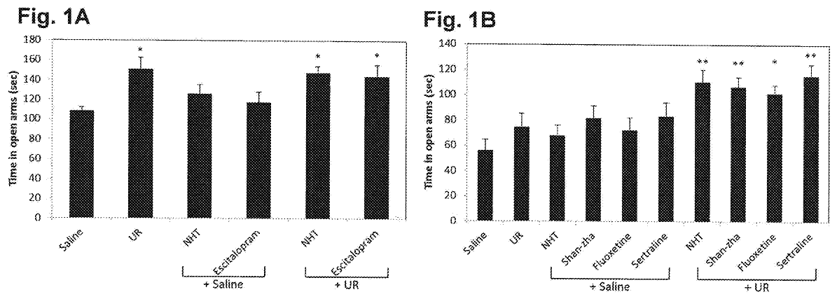

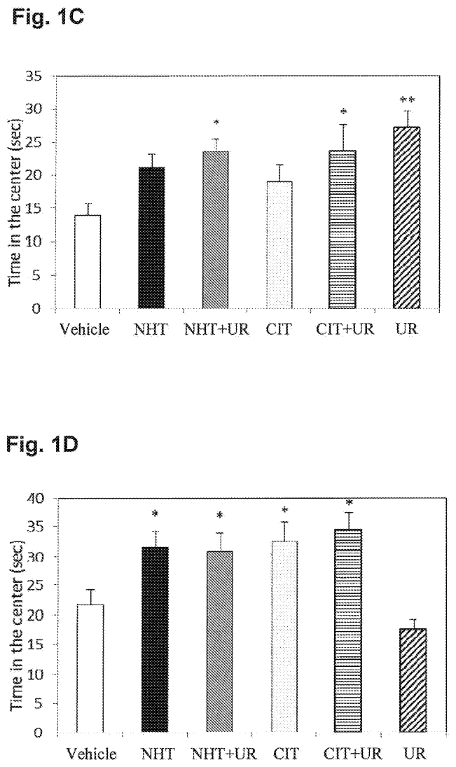

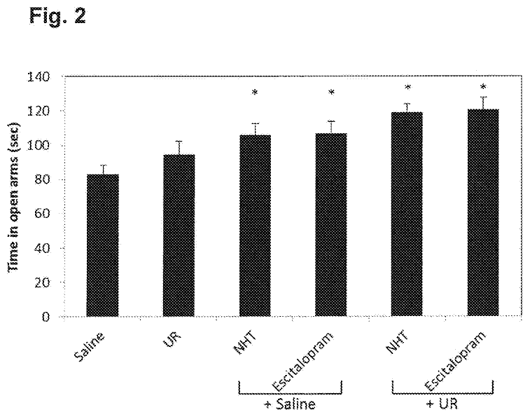

The anxiolytic-like effect in the Elevated Plus Maze test (EPM) and alterations in BDNF levels of 1- and 3-week combined UR treatments were evaluated compared to saline. In the first experiment, 1-week treatment with UR in combination with escitalopram and NHT (New Herbal Treatment—a herbal composition comprising four components: hawthorn fruit (Shan Zha), light wheat grain (Fu xiao mai), Lilly Bulb (bai hi) and Chinese date (Da zao)) reduced anxiety-like behavior (F(5,78)=3.229, p<0.011; p<0.025 and p<0.014, respectively) (FIG. 1A). These findings are supported by the second experiment which demonstrated the fast onset anxiolytic-like effects of 1-week treatment with UR in combination with the SSRIs fluoxetine and sertraline, NHT and Shan-zha (F(9,139)=4.780, p<0.001; p<0.002, p<0.001, p<0.001 and p<0.001, respectively) (FIG. 1B). Inconsistent effects were observed following UR individual treatment in the two experiments. Nevertheless, the combined treatments showed sustained effects. At 3 weeks, the combined UR treatments with NHT and escitalopram as well as the individual corresponding treatments reduced anxiety-like behavior (F(5,75)=4.624, P<0.001; p<0.05, p<0.05, p<0.001 and p<0.001, respectively) (FIG. 2). Biochemically, hippocampal BDNF levels were increased following 1-week combined UR treatments with escitalopram and NHT (F(5,59)=34.644, P<0.001; p<0.004 and p<0.001, respectively). Interestingly, in this time point individual treatments including UR reduced BNDF levels (p<0.005, p<0.008 and p<0.006, respectively) (FIG. 3A). At 3 weeks, only individual treatments with NHT and escitalopram increased BDNF levels as opposed to the combined treatments [F (5,43)=2.786, P<0.029] (p<0.050 and p<0.026, respectively) (FIG. 3B).

### Example 2

### Analysis of Anxiety-Like Behavior Using the Open Field Test (OFT)

In addition, the anxiety-like behavior was analyzed using the Open field test (OFT) following treatment with UR or in combination with NHT or escitalopram. It was observed that 1-week treatment with UR alone and in combination with NHT and escitalopram reduced anxiety-like behavior in the open field test (OFT) [F(5,78)=3.402, P<0.008] (FIG. 1C). At this early time point, treatments with NHT and escitalopram alone were ineffective. Following 3 weeks, all treatments, apart from UR, induced anxiolytic effect in the OFT [F(5,74)=6.392, P<0.001] (FIG. 1D). The behavioral findings support the notion that UR has the potential to induce a fast-onset anxiolytic effect when administered alone or in combination with other drugs.

### Example 3

### Analysis of Anxiety-Like Behavior Using the Novel Open Field (NOF)

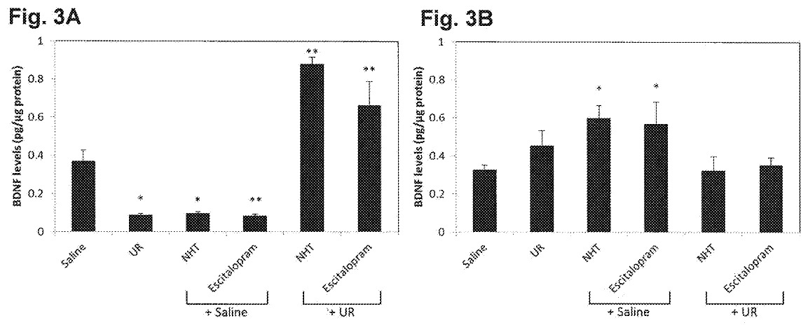

Anxiety-like behavior was evaluated in mice previously exposed to 30 days of unpredictable chronic mild stress paradigm, following 1 and 3 weeks of treatment in independent groups with either Escitalopram, NHT, UR, Escitalopram+UR, NHT+UR, Shan-Zha (SZ)+UR, or Saline. Mice treated with either the SZ+UR, NHT+UR, Ecitalopram+UR, and UR spent significantly more time in the center of the arena in comparison to the NHT, Escitalopram, Saline groups following 1 week treatment (FIG. 4A) (F(6,98)=2.31, p<0.05). Moreover, following 3 weeks of treatment, mice treated with either the NHT+UR, Ecitalopram+UR, NHT, or Escitalopram, spent significantly more time in the center of the arena in comparison to the Saline and UR groups (FIG. 4B) (F(5,77)=5.796, p<0.0001).

### Example 4

### Analysis of Anxiety-Like Behavior Using the Forced Swim Test (FST)

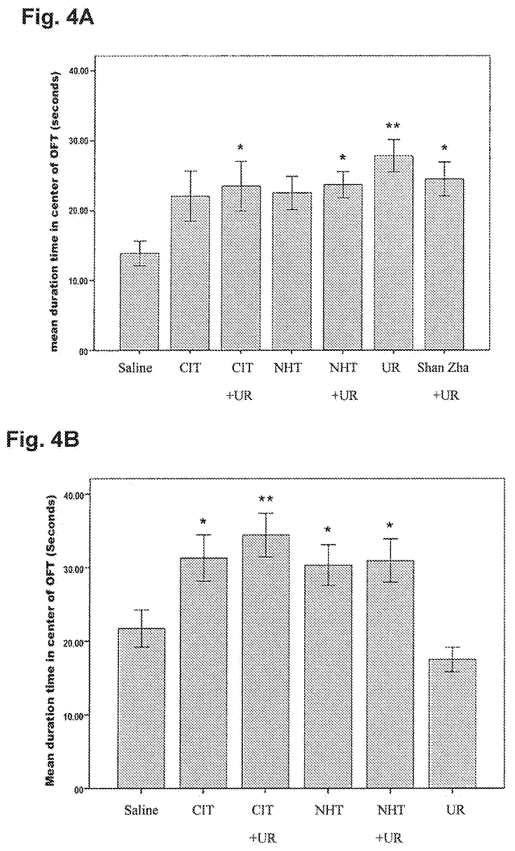

Depressive like behavior was evaluated in the FST in mice previously exposed to 30 days of unpredictable chronic mild stress paradigm, following 1 and 3 weeks of treatment in independent groups with either Escitalopram, NHT, UR, Escitalopram+UR, NHT+UR, SZ+UR, Saline. Interestingly, only mice treated with SZ+UR spent significantly less time floated-immobile, and exhibited reduced depressive-like behavior in the FST in 1 week treatment in comparison to NHT+UR, Ecitalopram+UR, NHT, Ecitalopram, UR, Saline (FIG. 5A) (F(6,95)=2.058, p<0.05). In contrast, Mice treated 3 weeks, with either the, NHT+UR, Ecitalopram+UR, Escitalopram and of UR spent significantly less time floated-immobile in the FST in comparison to the NHT and Saline groups following 3 week treatment (FIG. 5B) (F(5,71)=2.711, p<0.05).

### Example 5

### Evaluation of Side Effects of a Combination of Escitalopram or Shan-Zha With UR

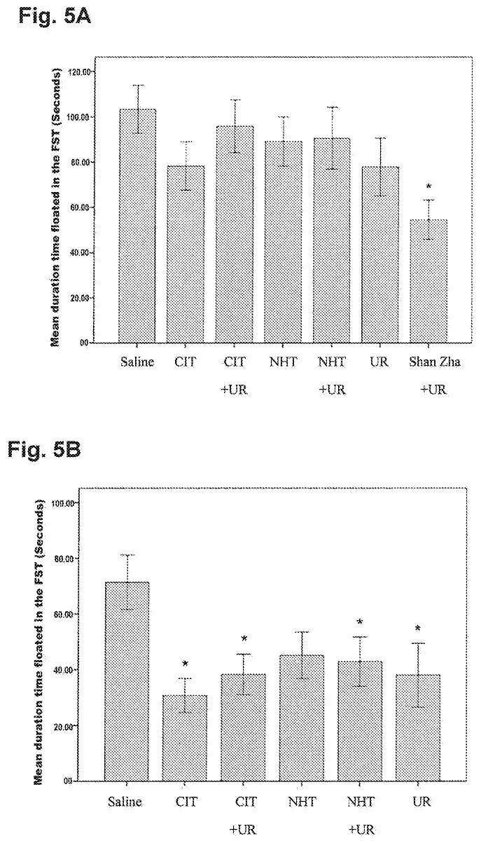

A common side-effect of SSRI drugs is sexual dysfunction. In addition to accelerating the therapeutic effect of escitalopram, the appearance of side-effects was also prompt. Mice treated with escitalopram+UR took longer time before exhibiting mounting behavior (FIG. 6A). On the contrary, mice treated with Shan-zha+UR did not show this side effect (FIG. 6B). These results are in accordance with previous data, showing the 3-week treatment with the Shan-zha herb effectively reduced anxiety-like behavior while precluding sexual dysfunction. These results demonstrate that the Uncaria rhynchophylla (UR) herb hastes the anxiolytic effect of Shan-zha and conventional drugs following one-week treatment in both anxiety and depression-like behavioral tests.

### Example 6

### Treatment of Human Subjects With UR in Combination With SSRIs

A clinical study is conducted for determining the on-set of alteration in depressive symptoms induced by treatment with UR in combination with SSRIs. Patients who are diagnosed with severe to moderate major depression disorder (preferably patients that are about to receive treatment with SSRI for the first time) are randomized to two groups (30 subjects aged 18-65 in each group). One group receiving an SSRI drug, e.g., escitalopram (10 mg)+placebo and the other group receiving the same SSRI drug, e.g., escitalopram (10 mg) +UR (450 mg) daily treatment or 0.5 g twice daily for two weeks followed by their regular treatment (without placebo or UR). Overall, the study is conducted for 6 weeks, and the patients are optionally monitored for 3 months. After baseline evaluation and signing informed consent, subjects are asked to fill a socio-demographic questionnaire, and to undergo a clinical differential diagnosis using the Symptoms Check List (SCL)-90, Clinical Global Impression (CGI), Hamilton anxiety rating scale (HAM-A), Hamilton depression rating scale (HAM-D), Sheehan Disability Scale (SDS), and two visual analogue scales (VAS)-general anxiety, and mood. Subjects are monitored at baseline and every two weeks for three months, using HAM-A, HAM-D, VASs and SDS. Adverse effects are documented every evaluation using the Treatment Emergent Symptom Scale. Various physiological parameters of the subjects are monitored, e.g., weight, heart rate and blood pressure at baseline and every follow-up meeting till the end of the trial. The UR and escitalopram are added to the current drug treatment (non-antidepressant). Patients are asked to provide a blood sample at the beginning and the end of the trial.

### Example 7

### Activation of GIRK (G Protein Activated Inward Rectifying K+) Channel by Uncaria

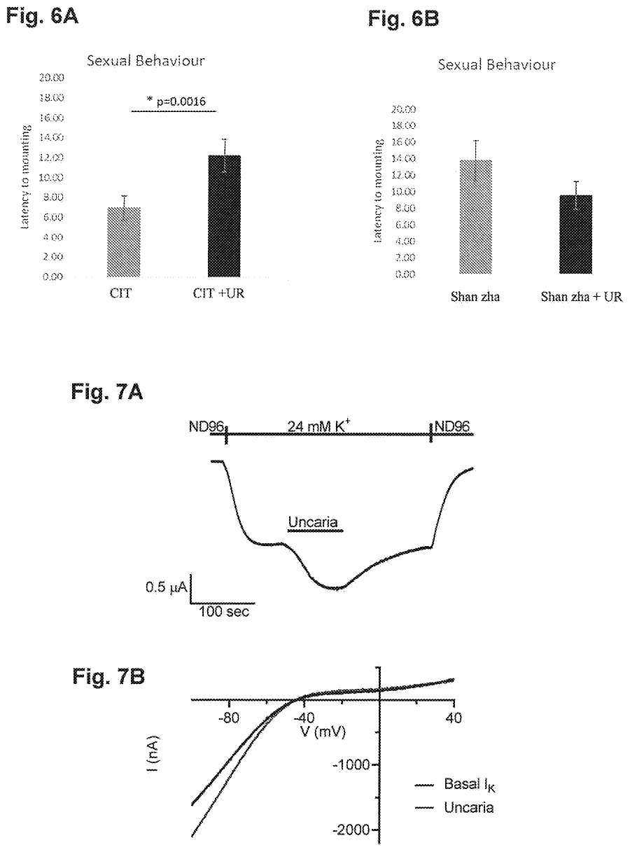

Two subunits of the GIRK channel (GIRK1 and GIRK2) were expressed in Xenopus oocytes. This was done by injecting 500 pg cRNA of the of the respective GIRK subunits (transcribed in vitro using a standard protocol) to oocytes. Three days after injection of the cRNA, the GIRK channel was expressed on the cell membrane of the oocyte and the following two electrode voltage clamp experiments were conducted: The oocyte was voltage clamped to −80 mV at 2 mM K+ (ND96) solution. Upon replacement to 24 mM K+ solution a basal K+ current evolved, which was evident by a large inward current. Then, Uncaria (2 g/liter) was applied and resulted in additional K+ current, indicating activation of the GIRK channel by Uncaria (FIG. 7A). The current-voltage relationship (FIG. 7B) is typical for GIRK activation, further supporting the conclusion that Uncaria induces activation of the channel.

Application of Uncaria to control, un-injected oocytes (oocytes that did not express the GIRK channels) had no effect when the same experimental procedure was repeated.

1. (canceled)

2. A method of treating or preventing anxiety, stress, depression, and/or symptoms thereof, or enhancing the rate at which an antidepressant or anxiolytic drug or an antidepressant or anxiolytic therapy affects the symptoms of anxiety, stress, and/or depression in a subject, said method comprising conjointly administering an Uncaria rhynchophylla (UR) herb and at least one antidepressant or anxiolytic drug or an antidepressant or anxiolytic therapy to a subject in need thereof.

3. (canceled)

4. The method of claim 1 wherein said stress is chronic stress or acute stress.

5. The method of claim 1 wherein said depression is Major Depressive Disorder (MDD).

6. The method of claim 1 wherein said administration is during a mild, moderate, or severe depressive episode.

7.-9. (canceled)

10. The method of claim 1 wherein the UR is administered in an amount of 450 mg per day, 0.5 g per day, 1 g per day, 2 g per day or more.

11. The method of claim 1 wherein the UR is administered once a day, twice a day, three times a day or more.

12. The method of claim 1 wherein said antidepressant or anxiolytic drug is selected from the group consisting of a serotonin modulator, a Selective serotonin reuptake inhibitor (SSRI), a serotonin-norepinephrine reuptake inhibitor, a herbal composition and a combination thereof.

13. The method of claim 7 wherein said serotonin modulator is vilazodone and/or wherein said Selective serotonin reuptake inhibitor is any one of escitalopram, fluoxetine, or sertraline, and/or wherein said serotonin-norepinephrine reuptake inhibitor is imipramine.

14-15. (canceled)

16. The method of claim 7 wherein said herbal composition is selected from a group consisting of Shan Zha, an active fraction of Shan Zha, and a combination of hawthorn fruit (Shan Zha), light wheat grain (Fu xiao mai), Lilly Bulb (bai hi) and Chinese date (Da zao).

17. The method of claim 1 wherein said conjoint administration comprises administering the UR and the at least one antidepressant or anxiolytic drug in a single composition or in separate compositions.

18. The method of claim 1 wherein said antidepressant or anxiolytic therapy is selected from the group consisting of psychotherapy, cognitive behavioral therapy (CBT), ECT (electroconvulsive therapy), transcranial direct current stimulation (tDCS), transcranial magnetic stimulation (TMS), DBS (deep brain stimulation), or physical exercise.

19. The method of claim 1 wherein said conjoint administration reduces the time required for reduction of anxiety, stress and/or depression symptoms.

20. The method of claim 12 wherein said symptoms are evaluated using a Symptoms Check List (SCL)-90, Clinical Global Impression (CGI), Hamilton anxiety rating scale (HAM-A), Hamilton depression rating scale (HAM-D), Sheehan Disability Scale (SDS), or visual analogue scales (VAS).

21. The method of claim 13 wherein said VAS are general anxiety or mood.

22. The method of claim 1 wherein said conjoint treatment is for 1 day, 2 days, 3 days, 4 days, 5 days, 6 days, 1 week, 2 weeks, 3 weeks, 4 weeks, 5 weeks, 6 weeks or more.

23. The method of claim 1 wherein said conjoint treatment does not cause weight gain, reduced sexual function, and/or reduced memory function.

24.-26. (canceled)

27. A composition comprising Uncaria rhynchophylla (UR) herb and at least one antidepressant or anxiolytic drug, said composition optionally further comprising at least one physiologically acceptable carrier, diluent, excipient and/or additive.

28. The composition of claim 17, wherein said composition is a pharmaceutical composition or herbal supplement.

29. (canceled)

30. A kit comprising Uncaria rhynchophylla (UR) herb and at least one antidepressant or anxiolytic drug, said kit optionally further comprising instructions for conjoint use of the UR and the at least one antidepressant or anxiolytic drug in treating or preventing anxiety, stress, depression, and/or symptoms thereof.

31. A method of rapid-onset treatment or prevention of anxiety, stress, depression, and/or symptoms thereof, said method comprising administering the Uncaria rhynchophylla (UR) herb as a single agent.
